(19) United States
(12) Patent Application Publication
(10) Pub. No.: US 2025/0025343 A1
(43) Pub. Date: Jan. 23, 2025

(54) METHOD FOR PROVIDING CONTROL DATA FOR AN OPTHALMOLOGICAL LASER OF A TREATMENT APPARATUS

(71) Applicant: SCHWIND EYE-TECH-SOLUTIONS GMBH, Kleinostheim (DE)

(72) Inventor: Samuel ARBA MOSQUERA, Aschaffenburg (DE)

(21) Appl. No.: 18/777,839

(22) Filed: Jul. 19, 2024

(30) Foreign Application Priority Data
Jul. 20, 2023 (DE) 10 2023 119 211.6

(51) Int. Cl. A61F 9/008

(52) U.S. Cl. CPC A61F 9/00814; A61F 2009/00855; A61F 2009/00882

(57) ABSTRACT

A method for providing control data for an ophthalmological laser of a treatment apparatus for treating a cornea, which has been treated with a cross-linking method. The method includes setting conventional laser parameters for removing a predetermined correction volume from the cornea, wherein the laser parameters include at least a laser pulse energy and a number of laser pulses of the laser; providing laser parameters increased in power in areas of the correction volume, in which a cornea changed by the cross-linking method is expected, wherein the laser parameters increased in power have a higher laser pulse energy and/or a higher number of laser pulses compared to the conventional laser parameters; and providing the control data, which includes the laser parameters increased in power in the areas of the correction volume with changed cornea and the conventional laser parameters in the remaining areas of the correction volume.

### FIELD

The invention relates to a method for providing control data for an ophthalmological laser of a treatment apparatus for treating a cornea, which has been treated with a cross-linking method. Furthermore, the invention relates to a control device, which is configured to perform the method, to a treatment apparatus with such a control device, to a computer program comprising commands, which cause the treatment apparatus to execute the method, and to a computer-readable medium, on which the computer program is stored.

### BACKGROUND

Treatment apparatuses and methods for controlling ophthalmological lasers for correcting an optical visual disorder and/or pathologically or unnaturally altered areas of the cornea are known in the prior art. Therein, pulsed lasers and a beam focusing device may, for example, be formed such that laser pulses effect a photodisruption and/or ablation in a focus area situated within the organic tissue to remove a tissue, in particular a tissue lenticule, from the cornea.

Furthermore, it is known to perform so-called cross-linking methods (cross-linking; CXL) for the cornea. Herein, the cornea is irradiated with UV light in particular with the aid of riboflavin (vitamin B2), which results in a cross-linking of the conjunctive tissue fibers and thus a structure of the cornea is reinforced to, for example, stabilize a keratoconus. However, a treatment with such a cross-linking method also influences a laser pulse interaction with the treated tissue of the cornea, whereby subsequent treatments with ophthalmological lasers are influenced and possibly a tissue separation is influenced.

### SUMMARY

It is the object of the present invention to improve a provision of control data for corneas, in which a cross-linking method has already been performed.

This object is solved by the independent claims. Advantageous embodiments of the invention are disclosed in the dependent claims, the following description as well as the figures.

An aspect of the invention relates to a method for providing control data for an ophthalmological laser of a treatment apparatus for treating a cornea, which has been treated with a cross-linking method, wherein the method comprises the following steps performed by a control device. Therein, an appliance or an appliance component, in particular a computer or processor, which may automatically or semi-automatically perform the following steps, is meant by a control device.

In the method, setting conventional laser parameters for removing a predetermined correction volume from the cornea, wherein the laser parameters include at least a laser pulse energy and a number of laser pulses of the laser, providing laser parameters increased in power in areas of the correction volume in which a change of the cornea by the cross-linking method is expected, wherein the laser parameters increased in power have a higher laser pulse energy and/or a higher number of laser pulses compared to the conventional laser parameters, and providing the control data, which includes the laser parameter increased in power in the areas of the correction volume with changed cornea and the conventional laser parameters in the remaining areas of the correction volume, are effected. That is, by areas of the correction volume in which a change of the cornea by the cross-linking method is expected, it is meant that areas of the cornea are estimated, measured, ascertained, or otherwise determined to be changed by the previous cross-linking method.

In other words, it may be provided that a cornea is treated, in which a cross-linking method has already been performed, and thus the cornea has changed areas by the cross-linking method. For planning the treatment with the treatment apparatus, therein, a predetermined correction volume may be set, which is to be treated with conventional laser parameters, wherein it may be examined if areas are present in the predetermined correction volume, in which the cornea has been changed by the cross-linking method. If this is the case, laser parameters increased in power may be planned for these changed areas compared to the areas in which a changed cornea is not present in the correction volume.

The power increase of the laser parameters may be achieved by an increase of the laser pulse energy and/or by a higher number of laser pulses in these areas. The higher number of laser pulses may be effected by an increase of the repetition rate on the one hand and/or by a reduction of a pulse distance of the laser pulses in these areas on the other hand. In particular, it is provided that the laser parameters increased in power provide a power increased at least by 10% compared to the conventional laser parameters.

In particular, the cornea may have a higher stability in areas, which have been changed by the cross-linking method, whereby a higher energy for separating corneal layers is required. Herein, the laser parameters increased in power do not have to be increased to a constant value, but may be adapted depending on an assumed size or of the change by the cross-linking method. For all of the further areas of the cornea, conventional laser parameters may be used, wherein the conventional laser parameters may be optimized to a (photo) ablation and/or photodisruption of the unchanged corneal tissue.

The areas in which the change by the cross-linking method is expected, may, for example, be estimated, in particular based on diffusion models of the riboflavin in the cornea and/or absorption gradients of the UV light in the cornea. Alternatively or additionally, the localization of the changed cornea may also be determined by measurements of the cornea before the treatment.

Finally, the control data may be provided, in which the areas, which are irradiated with laser parameters increased in power, and areas, in which the conventional laser parameters are used, are set.

The control data may include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser may be included in the control data.

By the invention, the advantage arises that a treatment of corneas, which have already been treated with a cross-linking method, is improved.

The invention also includes embodiments, by which additional advantages arise.

In an embodiment, change by the cross-linking method may be present from a surface of a stroma of the cornea down to a preset depth in the cornea. This means that the cross-linked areas are assumed from an anterior interface of the stroma in depth direction or z-direction down to a preset depth in the cornea, which may also be situated in the stroma. For example, the preset depth may be preset up to a depth value of 100 μm, in particular 50 μm, in the stroma of the cornea. These values represent preferred depths, in which the cornea is still changed by the cross-linking method, wherein 50 μm are assumed in a conventional cross-linking method, in which additional riboflavin is not applied, and 100 μm in an accelerated cross-linking method, in which riboflavin is applied. The epithelial layer, which is above the stroma, may not be treated by the laser parameters increased in power, but by the conventional laser parameters.

In another embodiment, a gradient of the changed cornea is assumed down to the preset depth in the cornea, wherein the laser parameters increased in power are provided depending on the gradient. This means that the cornea is severely cross-linked and thus changed in the upper corneal layers, wherein the cross-linking decreases with increasing depth. Accordingly, in positions of the upper corneal layer, laser parameters increased in power with a high laser pulse power may be provided, wherein the laser pulse power may be reduced with increasing depth in the cornea corresponding to the gradient, until it finally reaches the conventional laser parameters in the preset depth. This means that the laser power may decrease with increasing depth along the gradient of the cross-linking. Hereby, the advantage arises that suitable laser parameters may be provided for each degree of the cross-linking.

In a further embodiment, the change of the cornea by the cross-linking method is assumed as a gradient in the radial direction, wherein a higher degree of a cross-linking is assumed in a center of the cornea than in a periphery of the cornea, wherein the laser parameters increased in power are provided depending on the gradient in the radial direction. Thus, a diffusion of the riboflavin from the inside to the outside may for example occur in the cross-linking method, whereby a higher degree of the cross-linking is present in the center of the cornea than in the periphery. Correspondingly, the laser parameters may be adjusted such that the laser parameters increased in power with the greatest power are provided in the center, which are reduced outwards along the radial direction, until they finally reach the laser pulse power of the conventional laser parameters. In particular, the previously mentioned gradient may be combined with the gradient in the radial direction in the direction of the depth of the cornea to thus provide the optimized laser parameters depending on the z-direction and depending on the radial direction, to irradiate the cornea changed by the cross-linking method.

In a further embodiment, the areas in which the change by the cross-linking method is expected are determined from predetermined measurement data, in particular by a Brillouin spectroscopy. In other words, the areas in which the laser parameters increased in power are to be provided may be predetermined by measurements. In these areas, which are determined by measurement, constant laser parameters increased in power may be provided, for example, increased by 10% compared to the conventional laser parameters, or the laser parameters increased in power may be provided depending on a degree of the cross-linking. A preferred possibility of ascertaining a degree of the cross-linking is a Brillouin spectroscopy, by which Young's moduli of materials may be determined in that the inelastic scattering of light on acoustic photons is examined. By this embodiment, the advantage arises that the areas with changed or cross-linked cornea may be most accurately localized.

In a further embodiment, a warning signal is generated if it is ascertained that a removal of the cornea changed by the cross-linking method exceeds a preset threshold value by the generation of the correction volume. In other words, a warning signal may be provided if it is ascertained that too much cross-linked tissue is to be removed from the cornea by the removal of the correction volume. For example, the warning signal may be output to a user via a user interface, for example, via a display device or an acoustic device. Herein, a comparison may occur by the control device of how large the correction volume is and how many percent of the cornea changed by the cross-linking method is in the correction volume. By this embodiment, a precautional measure may be provided that not too much of the cross-linked cornea is removed since a stability of the entire cornea could thus be impaired.

In a further embodiment, the areas in which the change by the cross-linking method is expected are ascertained depending on a previously known duration of the cross-linking method and/or a previously known treatment performance of the cross-linking method. In other words, it may be estimated, in which areas the cornea is cross-linked from the previous cross-linking method and/or how severely the cornea is cross-linked in areas, if parameters of the used cross-linking method are known. In particular, a duration of the cross-linking method may be decisive to what extent the riboflavin has penetrated into the cornea and thus down to which depth the cross-linked cornea is to be expected. Thus, the duration may, for example, include 5 to 45 minutes according to used cross-linking method. A differentiation if a conventional cross-linking method, an accelerated cross-linking method, in which additional pharmaceuticals are administered for acceleration of a diffusion or osmosis, or a cross-linking method, in which an epithelial layer remains present, may also be used. By this embodiment, the advantage arises that an improved localization of the cross-linked cornea may be performed.

A further aspect relates to a method for controlling a treatment apparatus. Therein, the method includes the method steps of at least one embodiment of a method as it was previously described. Furthermore, the method for controlling the treatment apparatus also includes the step of transferring the provided control data to at least one ophthalmological laser of the treatment apparatus and controlling the treatment apparatus and/or the laser with the control data.

The respective method may include at least one additional step, which is executed if and only if an application case or an application situation occurs, which has not been explicitly described here. For example, the step may include the output of an error message and/or the output of a request for inputting a user feedback. Additionally or alternatively, it may be provided that a default setting and/or a predetermined initial state are adjusted.

A further aspect of the invention relates to a control device, which is formed to perform the steps of at least one embodiment of one or both of the previously described methods. Thereto, the control device may comprise a computing unit for electronic data processing such as, for example, a processor. The computing unit may include at least one microcontroller and/or at least one microprocessor. The computing unit may be configured as an integrated circuit and/or microchip. Furthermore, the control device may include an (electronic) data memory or a storage unit. A program code may be stored on the data memory, by which the steps of the respective embodiment of the respective method are encoded. The program code may include the control data for the respective laser. The program code may be executed by the computing unit, whereby the control device is caused to execute the respective embodiment. The control device may be formed as a control chip or control unit. The control device may, for example, be encompassed by a computer or computer cluster.

A further aspect of the invention relates to a treatment apparatus with at least one eye surgical or ophthalmological laser and a control device, which is formed to perform the steps of at least one embodiment of one or both of the previously described methods. The respective laser may be formed to at least partially separate a predefined corneal volume with predefined interfaces of a human or animal eye by optical breakdown, in particular at least partially separate it by photodisruption and/or to ablate corneal layers by (photo) ablation and/or to effect a laser-induced refractive index change in the cornea and/or the eye lens.

In a further embodiment of the treatment apparatus according to the invention, the laser may be suitable to emit laser pulses in a wavelength range between 300 nm and 1400 nm, for example, between 900 nm and 1200 nm, at a respective pulse duration between 1 fs and 1 ns, for example, between 10 fs and 10 ps, and a repetition frequency of greater than 10 kilohertz (kHz), for example, between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakdown may be spatially narrowly limited such that a high incision accuracy is allowed in the generation of the interfaces. In particular, the range between 700 nm and 780 nm may also be selected as the wavelength range.

In a further embodiment of the treatment apparatus according to the invention, the control device may comprise at least one storage device for at least temporary storage of at least one control dataset, wherein the control dataset or datasets include(s) control data for positioning and/or for focusing individual laser pulses in the cornea; and may comprise at least one beam device for beam guidance and/or beam shaping and/or beam deflection and/or beam focusing of a laser beam of the laser.

A further aspect of the invention relates to a computer program. The computer program includes commands, which, for example, form a program code. The program code may include at least one control dataset with the respective control data for the respective laser. Upon execution of the program code by a computer or a computer cluster, it is caused to execute the previously described method or at least one embodiment thereof.

A further aspect of the invention relates to a computer-readable medium (storage medium), on which the above-mentioned computer program and the commands thereof, respectively, are stored. For executing the computer program, a computer or a computer cluster may access the computer-readable medium and read out the content thereof. The storage medium is, for example, formed as a data memory, in particular at least partially as a volatile or a non-volatile data memory. A non-volatile data memory may be a flash memory and/or an SSD (solid state drive) and/or a hard disk. A volatile data memory may be a RAM (random access memory). For example, the commands may be present as a source code of a programming language and/or as assembler and/or as a binary code.

Further features and advantages of one of the described aspects of the invention may result from the embodiments of another one of the aspects of the invention. Thus, the features of the embodiments of the invention may be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following may be present in any combination with each other and/or the features of the embodiments. This means, the features of the execution examples may supplement and/or replace the features of the embodiments and vice versa. Thus, configurations are also to be regarded as encompassed and disclosed by the invention, which are not explicitly shown or explained in the figures, but arise from and may be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 depicts a schematic representation of a treatment apparatus according to an exemplary embodiment.

FIG. 2 depicts a schematic method diagram for providing control data according to an exemplary embodiment.

In the figures, identical or functionally identical elements are provided with the same reference characters.

### DETAILED DESCRIPTION

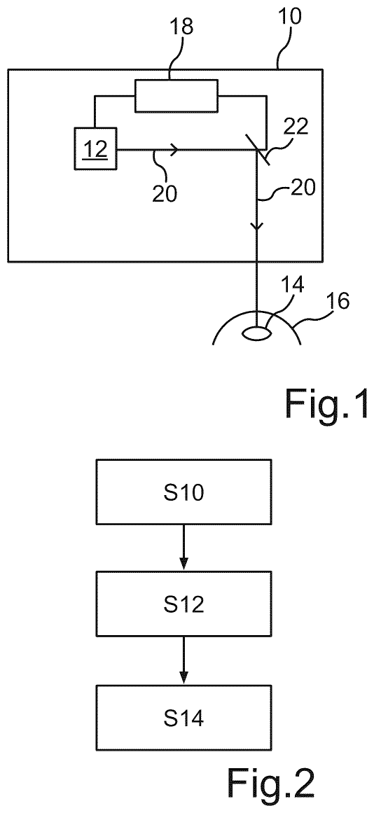

FIG. 1 shows a schematic representation of a treatment apparatus 10 with an ophthalmological laser 12 for removing a correction volume 14 from a human or animal cornea 16 by photodisruption and/or ablation. The correction volume 14 may, for example, represent a lenticule or also volume body, which may be separated from the cornea 16 with the eye surgical laser 12 for correcting a visual disorder. The correction volume 14 or a geometry of the correction volume 14 to be removed may be preset to a control device 18, in particular in the form of control data, such that the laser 12 emits pulsed laser pulses in a pattern predefined by the control data into the cornea 16 of the eye to remove the correction volume 14. Alternatively, the control device 18 may be a control device 18 external with respect to the treatment apparatus 10.

Furthermore, FIG. 1 shows that the laser beam 20 generated by the laser 12 may be deflected towards the cornea 16 by a beam deflection device 22 such as, for example, a rotation scanner, to remove the correction volume 14. The beam deflection device 22 may also be controlled by the control device 18 to remove the correction volume 14.

In particular, the illustrated laser 12 may be a photodisruptive and/or photoablative laser, which is formed to emit laser pulses in a wavelength range between 300 nanometers and 1400 nanometers, for example, between 700 nanometers and 1200 nanometers, at a respective pulse duration between 1 femtosecond and 1 nanosecond, for example, between 10 femtoseconds and 10 picoseconds, and a repetition frequency of greater than 10 kilohertz, for example, between 100 kilohertz and 100 megahertz. In addition, the control device 18 optionally comprises a storage device (not illustrated) for at least temporary storage of at least one control dataset, wherein the control dataset or datasets include(s) control data for positioning and/or for focusing individual laser pulses in the cornea.

If the cornea 16 has been treated with a cross-linking method in a pretreatment, areas of the cornea 16 may be changed by the cross-linking and thus have a tighter structure than non-treated areas. Herein, the characteristics of the cornea 16 also change with respect to the separation of the correction volume 14 such that a generation of the correction volume 14 with conventional laser parameters is impaired. In order to consider the effect of a preceding cross-linking method, therefore, the control device 18 may perform the method shown in FIG. 2.

In FIG. 2, a schematic method diagram for providing control data for an ophthalmological laser 12 of a treatment apparatus 10 for treating a cornea 16 is illustrated, wherein the cornea 16 has previously been treated with a cross-linking method.

In a step S10, conventional laser parameters for removing a predetermined correction volume 14 from the cornea 16 may be provided. For example, the correction volume may be set for correcting a visual disorder and/or an unnaturally or pathologically altered tissue of the cornea 16 based on predetermined examination data. Hereto, conventional laser parameters, which in particular include a laser pulse energy and a number of laser pulses per tissue volume, may be planned, which are optimized for usual corneal tissue. This means that an ablation of corneal tissue and/or incisions in the corneal tissue may be performed with the conventional laser parameters without the development of disadvantageous effects, such as, for example, an opaque bubble layer or black spots.

In a step S12, laser parameters increased in power may be planned in areas of the correction volume 14 in which a change to the cornea by the cross-linking method is expected, estimated, ascertained, or otherwise determined to be present. Herein, the laser parameters increased in power may have a higher laser pulse power than the conventional laser parameters, wherein the laser pulse energy may in particular be increased hereto and/or a higher number of laser pulses per volume may be planned. For increasing the number of laser pulses, a repetition rate of the laser and/or a laser pulse distance in the corresponding areas may, for example, be changed. The laser parameters increased in power may either be constantly increased compared to the conventional laser parameters, for example, by 10%, or the laser parameters increased in power may be adapted depending on a degree of the change. This means that the higher the cornea is cross-linked, the higher the laser pulse power may be adjusted, wherein the degree of the cross-linking and thus the adjusted laser pulse power may be different depending on the position in the cornea 16.

In the step S12, it may be previously determined where the areas of the cornea changed by the cross-linking method are located. Furthermore, a degree of the change may herein be determined. In order to determine the areas with increased cross-linking, either a measurement may be performed, in particular with a Brillouin spectroscopy, or the areas may be estimated by a model.

In estimating by the model, a previously known treatment performance of the cross-linking method, in particular with a known duration, may be used on the one hand to ascertain the areas with changed cornea. Herein, it may be assumed that the cornea 16 has a higher cross-linking on a surface, that is in the direction of the treatment apparatus 10, than in a depth, wherein a gradient, that is a decrease from the surface into the depth, is in particular present. Thus, the laser parameters increased in power may, for example, be selected higher on a surface than in a depth direction, wherein the laser parameters increased in power may decrease depending on the gradient of the depth, for example, down to a preset depth of 100 μm, in particular 50 μm, of the stroma, on which an effect of the cross-linking is no longer expected.

In corresponding manner, a gradient of the cross-linking in the radial direction may be planned such that a higher degree of the cross-linking is assumed in central areas than in a periphery. In other words, a laser power in the laser parameters increased in power may decrease from a surface towards the depth and from a center towards a periphery.

In a step S14, control data may then be generated, which has the laser parameters increased in power in the areas of the correction volume 14, which have been changed by the cross-linking method, and the conventional laser parameters may be used in the remaining areas of the correction volume 14. In particular, the laser pulse parameters increased in power may only be planned in the stroma of the cornea 16, wherein it may thus be provided in incisions or an ablation in the epithelium of the cornea 16 that the conventional laser parameters are set.

Overall, the examples show how an improved treatment planning may be provided for the cornea 16, which has been pretreated with a cross-linking method.

1. A method for providing control data for an ophthalmological laser of a treatment apparatus for treating a cornea, which has been treated with a cross-linking method, wherein the method comprises the following steps performed by a control device:
setting conventional laser parameters for removing a predetermined correction volume from the cornea, wherein the laser parameters include at least a laser pulse energy and a number of laser pulses of the laser;
providing laser parameters increased in power in areas of the correction volume, in which change to the cornea by the cross-linking method is expected, wherein the laser parameters increased in power have a higher laser pulse energy and/or a higher number of laser pulses compared to the conventional laser parameters; and
providing the control data, which includes the laser parameters increased in power in the areas of the correction volume in which change to the cornea by the cross-linking method is expected and the conventional laser parameters in remaining areas of the correction volume.

2. The method according to claim 1, wherein the change in the cornea by the cross-linking method is expected from a surface of a stroma of the cornea down to a preset depth in the cornea.

3. The method according to claim 2, wherein the change in the cornea is assumed as a gradient down to the preset depth in the cornea, wherein the laser parameters increased in power are provided depending on the gradient.

4. The method according to claim 2, wherein the preset depth is preset up to a depth value of 100 μm, in particular 50 μm, in the stroma of the cornea.

5. The method according to claim 1, wherein the change in the cornea by the cross-linking method is assumed as a gradient in a radial direction, wherein a higher degree of cross-linking is assumed in a center of the cornea than in a periphery of the cornea, wherein the laser parameters increased in power are provided depending on the gradient in the radial direction.

6. The method according to claim 1, wherein the areas in which change by the cross-linking method is expected are determined from predetermined measurement data.

7. The method according to claim 1, wherein a warning signal is generated if it is ascertained that the areas of the correction volume which have been changed by the cross-linking method exceeds a preset threshold value.

8. The method according to claim 1, wherein the areas in which change by the cross-linking method is expected, are ascertained depending on a previously known duration of the cross-linking method and/or a previously known treatment performance of the cross-linking method.

9. A control device, which is configured to perform a method according to claim 1.

10. A treatment apparatus with at least one eye surgical laser for separation of a corneal volume of a human or animal eye by optical breakdown, in particular by photodisruption and/or photoablation, and at least one control device according to claim 9.

11. (canceled)

12. A computer-readable medium for storing a computer program, the computer program comprising commands which cause a treatment apparatus to execute a method according to claim 1.

13. The method according to claim 6, wherein the areas in which change by the cross-linking method is expected are determined from a Brillouin spectroscopy.
